(19) United States
(12) Patent Application Publication
(10) Pub. No.: US 2002/0038049 A1
(43) Pub. Date: Mar. 28, 2002

(54) SULPHONATION PROCESS AND NOVEL SULPHONATED COMPOUNDS

(76) Inventors: JOHN HIBBS, CHESHIRE, GBN (US); DEBORAH ANN LEAH, LANCASHIRE, GBN (US); PAUL EVERETT, MANCHESTER, GBN (US)

Correspondence Address: RICHARD E JENKINS, JENKINS & WILSON, 3100 TOWER BOULEVARD, SUITE 1400 UNIVERSITY TOWER, DURHAM, NC, 27707, US

(21) Appl. No.: 09/463,375

(22) PCT Filed: May 24, 1999
(86) PCT No.: PCT/GB99/01452

(30) Foreign Application Priority Data
May 22, 1998 (GB) 9810946.5

(51) Int. Cl. C07C 309/00

(52) U.S. Cl. 562/98; 562/99

(57) ABSTRACT

An improved process for sulphonation of organic compounds, notably hydroxybenzene and alkylbenzene compounds, comprises reacting them with sulphuric acid or oleum in the presence of an organic solvent, notably an n-alkane or a cycloalkane. Also described is a method of extracting an alkylbenzene sulphonic compound from a sulphonation reaction mixture, using an alkylbenzene extractant of lower molecular weight, for example cumene. Some of the alkylbenzene sulphonic compounds are novel.

This invention relates to a sulphonation process and to novel sulphonated compounds.

Existing processes for the preparation of aromatic sulphonates, for example alkylbenzene sulphonates, employ sulphur trioxide to introduce the sulphonic group —SO₃— into the aromatic ring. There are a number of possible processes. One employs sulphur trioxide in air. This is an efficient large-scale process for the sulphonation of higher alkylbenzene compounds, for example nonylbenzene and higher homologues. However it is not suitable for the sulphonation of low molecular weight alkylbenzene compounds because of their high evaporation rate and the risk of an explosion in the electrostatic precipitator which is needed. A second process employs sulphur trioxide in sulphuric acid or liquid sulphur dioxide. In sulphur dioxide this process can be operated at low temperatures giving clean products but has high capital and running costs. In sulphuric acid the process is cheaper but has the disadvantage of generating large volumes of waste acid. A third process employs sulphuric acid or oleum with an excess of the aromatic compound, under reflux conditions. The water of reaction is thereby removed and the reaction consumes all of the sulphuric acid or oleum. This process is commonly used for the sulphonation of low molecular weight alkylbenzene compounds, for example toluene, cumene and xylene. However the process may be restricted by the howling point of the aromatic compound.

All of the existing processes have drawbacks. Furthermore there is no viable way of manufacturing alkylbenzene sulphonates of intermediate molecular weight. Indeed, many such alkylbenzene sulphonates are believed to be novel.

We have now devised a novel process for the sulphonation of alkylbenzene compounds, including those of intermediate molecular weight. Furthermore, we believe our novel process to be suitable for introducing the sulphonate group —SO₃— into a wide range of organic compounds.

In accordance with a first aspect of the present invention there is provided a process for the sulphonation of an organic compound, which process comprises reacting the organic compound with sulphuric acid or oleum in the presence of an organic solvent at an elevated temperature.

By “sulphonation” in the context of this invention we mean introduction of one or more sulphonic groups —SO₃—; whether the resultant product is a sulphonic acid (R—SO₃—H), a sulphonate (R—SO₃—X) or a sulphuric acid ester (R—O—SO₃—X).

Later in this specification we refer to “optionally substituted” groups. Within the meaning of this specification an optionally substituted aromatic or heteroaromatic group is suitably such a group optionally substituted by one or more substituents independently selected from halogen, especially fluorine, chlorine and bromine atoms, and nitro, cyano, hydroxyl, C₁₋₈ alkyl, C₁₋₈ haloalkyl (especially CF₃), C₁₋₈ alkoxy, C₁₋₈ haloalkoxy, amino, C₁₋₄ alkylamino and di- (C₁₋₄ alkyl) amino groups. 1 to 3 substituents may suitably be employed. The amino and alkylamino groups are least preferred, of the above substituents. Hydroxyl and, especially, alkyl groups are especially preferred substituents. An optionally substituted aliphatic or alicyclic group is suitably such a group optionally substituted by one or more substituents independently selected from halogen, especially fluorine, chlorine or bromine atoms, and nitro, cyano, hydroxyl, C₁₋₄ alkoxy, C₁₋₄ haloalkoxy, (C₁₋₄ alkoxy)carbonyl, amino, C₁₋₄ alkylamino and di (C₁₋₄ alkyl) amino groups. When an aliphatic group is substituted 1-3 substituents may suitably be present. The amino and alkylamino groups are least preferred, of the above substituents. It is preferred, however, that aliphatic groups are unsubstituted.

By elevated temperature we mean a temperature above ambient temperature, suitably a temperature of at least 50° C. A preferred elevated temperature is at least 70° C. or more preferably at least 90° C.

Suitably the elevated temperature does not exceed 180° C., and preferably does not exceed 160° C. Most preferably the elevated temperature does not exceed 140° C.

Preferably the reaction is carried out with removal of water of reaction, as the reaction proceeds. This improves the yield by driving the equilibrium reaction towards completion.

A preferred reaction is carried out under reflux. Thus preferred temperatures for the reaction may be expressed an temperatures in the range between any of the lower temperatures mentioned above, up to the reflux temperature, which preferably does not exceed one of the higher temperatures mentioned above.

The reaction may suitably be carried out at atmospheric pressure or at elevated pressure, or at sub-atmospheric pressure, for example under vacuum conditions, A suitable pressure range in 0.1×10⁵ Pa-5×10⁵ Pa. The use of sub-atmospheric pressure or vacuum conditions has the advantage that the boiling point of the reaction mixture is reduced, thereby reducing unwanted side reactions. Preferred conditions may therefore be atmospheric conditions or reduced pressure (but not vacuum) conditions. A preferred pressure range is 0.5×10⁵ Pa-1.1×10⁵ Pa.

Suitably the molar ratio of the organic compound, to be sulphonated (A) to the sulphuric acid or oleum (B)—the molarity of oleum being expressed as equivalent sulphuric acid molarity—is 0.5-1.5 mole (A) per 1 mole (B); preferably 0.7-1.3 mole (A) Per 1 mole (B); more preferably still 0.8-1.2 mole (A) per 1 mole (B). Most preferably, the molar ratio is substantially stoichiometric, by which we mean 0.9-1.1 mole (A) per 1 mole (B). From the point of view of Purification an excess of (B) is preferred—that is, the molar ratio is suitably 1 mole (A) per 1-1.5, preferably 1-1.2, more preferably 1-1.3, and most preferably 1-1.1 mole (B).

Preferably, the solvent is present in the reaction mixture in an amount exceeding 15% preferably exceeding 25%, of the total weight of the reaction mixture.

Preferably the solvent is present in the reaction mixture in an amount not exceeding 70%, preferably in an amount not exceeding 50%, of the total weight of the reaction mixture.

Most preferably the solvent in present in the reaction mixture in an amount in the range 25-40%; of the by total weight of the reaction mixture,

The solvent, which may be one or more organic liquids, is preferably inert, by which we mean that it does not effectively compete for the sulphonating agent with the organic compound required to be sulphonated. Any solvent may conceivably be sulphonated to an extent under certain conditions but any sulphonation of the solvent should be insubstantial, compared with the sulphonation of the said organic compound. Further, the solvent should be such as to permit the process to be operated at the required temperature for the reacting selected; be chemically and physically compatible with the reactants; and permit adequate work-up of the reaction mixture at the end of the reaction. Preferred solvents are non-polar. Preferred solvents are substantially free of water at the start of the reaction.

Suitable as solvents are optionally substituted aliphatic and alicyclic hydrocarbons, for example optionally substituted alkanes and cycloalkanes. Preferred are haloalkanes or, most preferably, alkanes having 4-14 carbon atoms: suitably 6-12, preferably 7-10. preferred alkane solvents are n-alkanes. Preferred cycloalkanes have 4-14 carbon atoms, preferably 6-12. Preferably alkane and cycloalkane solvents are unsubstituted.

Especially preferred solvents are cyclohexane and, especially, n-heptane and n-octane.

Preferably, the sulphonating agent is concentrated sulphuric acid, preferably containing at least 90 wt % sulphuric acid, or, especially, oleum, that is, sulphuric acid containing sulphur trioxide in solution. A preferred sulphonating agent is sulphuric acid containing 5-40% by weight of sulphur trioxide, preferably 10-30% by weight of sulphur trioxide. Most preferred is sulphuric acid containing about 20% by weight of sulphur trioxide (i.e the product known as oleum 20%).

The organic compound to be sulphonated can be any organic compound which can undergo a sulphonation reaction. It may be an aromatic compound, for example containing a benzene or naphthalene group, or a compound having three or more fused rings; it may be a heteroaromatic compound; or it may be an aliphatic compound, for example an alkanol. Aromatic and aliphatic compounds are preferred.

In preferred embodiments, the organic compound to be sulphonated is an optionally substituted aromatic compound, for example optionally substituted naphthalene or, especially, optionally substituted benzene.

Certain preferred optionally substituted aromatic organic compounds to be sulphonated are hydroxybenzene compounds, in particular benzene compounds having 1, 2 or 3 hydroxyl groups, especially phenol.

Especially preferred optionally substituted aromatic organic compounds to be sulphonated are alkylbenzene compounds, in particular benzene compounds having one or two alkyl groups having in total at least four carbon atoms. Preferred examples are diethylbenzene, diisopropylbenzene and n-hexylbenzene.

The process of the invention offers particular advantages in the preparation of alkylbenzene compounds having one or more alkyl groups which have in total 4-6 carbon atoms, such compounds having been difficult or impossible to make heretofore.

In the case of hydroxybenzene and alkylbenzene compounds, and most benzene-containing compounds, the product has a sulphonic group —SO₃—, or sulphonic acid groups, bonded directly to the ring, usually predominantly at the ortho and/or para positions relative to substituent(s).

Other preferred compounds to be sulphonated are benzene compounds having 1 or more, preferably 1 or 2, alkyl groups and 1 or more, preferably 1 or 2, hydroxyl groups, for example cresols and xylenols.

Preferred aliphatic organic compounds to be sulphonated ate n-alkanols, preferably having 4-14, especially 5-9, carbon atoms. In the case of these compounds the sulphonation reaction produces compounds known informally as alkyl sulphates, which are more rigorously named as sulphuric acid esters (i.e. compounds with the formula (R—O—SO₃—X).

At the required point the sulphonation reaction may suitably be terminated by a water quench.

The direct product of the process of the present invention is an acid. This may be derivatised in the usual ways e.g. to a metal salt, especially an alkali metal salt, especially sodium or potassium, or to an optionally substituted ammonium salt, for example ammonium, an alkylammonium or an alkanolammonium salt.

In accordance with a second aspect of the present invention there is provided a method of extracting an alkylbenzene sulphonic compound from a reaction mixture containing unreacted alkylbenzene, wherein the benzene group carries 1 or more alkyl groups comprising in total n or more carbon atoms wherein n is at least 3, which process comprises contacting the reaction mixture with a second, purifying compound having 1 or more alkyl groups comprising in total no more than m carbon atoms, wherein n is 2 or more integers greater than m, the purifying compound being able to form a separate layer in the reaction mixture, wherein the purifying compound is provided in an amount sufficient to form a said separate layer; and physically separating the two layers to remove unreacted alkylbenzene partitioned within the purifying compound layer; optionally neutralising the sulphonic acid in the sulphonic layer; and optionally removing any alkylbenzene partitioned within the sulphonic layer, for example by distillation.

In relation to this second aspect of the invention preferably the benzene group of the reactant carries 1, 2 or 3 alkyl groups, preferably 1 or 2. Preferably the alkyl group (when there is 1) or the alkyl groups cumulatively (when there is more than 1) comprise(s) 3-14 carbon atoms, especially 4-8 carbon atoms, most preferably 6 carbon atoms. Especially preferred compounds are diisopropylbenzene and n-hexylbenzene.

The second, purifying, alkylbenzene compound preferably has one or two alkyl groups comprising in total no more than three carbon atoms. Examples of compounds suitable as the second, purifying, alkylbenzene compounds are cumene, xylene and toluene.

Preferably n is three or more integers greater than m.

In an alternative definition of the second aspect of the present invention there is provided a method of extracting a sulphonic compound from a reaction mixture containing the unreacted (non-sulphonated) precursor compound, which process comprises contacting the reaction mixture with a second, purifying, compound having a boiling point not exceeding 140° C. at atmospheric pressure, and preferably being capable of steam distillation, the purifying compound being able to form a separate layer in the reaction mixture, wherein the purifying compound is provided in an amount sufficient to form a said separate layer; and physically separating the two layers to remove unreacted precursor compound partitioned within :he purifying compound layer; optionally neutralising the sulphonic acid in the sulphonic layer; and optionally removing any precursor compound or purifying compound partitioned within the sulphonic layer, suitably by distillation.

In accordance with a third aspect of the present invention there is provided a novel compound of general formula

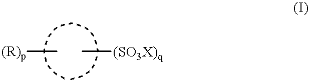

wherein the dotted ring symbol represents a heteroaromatic or, especially, an aromatic group; R represents, or each group R independently represents, a halogen atom, or a cyano, hydroxyl or amino group, or an optionally substituted alkyl, alkoxy, alkylamino or dialkylamino group;, p represents 1, 2, 3, 4 or 5; X represents, or each group X independently represents, a hydrogen or metal atom or an optionally substituted ammonium group; and q represents 1, 2 or 3; provided that (p+q)≦6.

Preferably the or each group R represents a hydroxyl and/or an alkyl group, as defined above in relation to the first aspect of the present invention. Preferably the or each group R represents an alkyl group Preferably the total number of carbon atoms in the alkyl group (when there is 1) or in all of the alkyl groups (when there is more than 1) is at least 4, but preferably no more than B.

Preferably p represents 1, 2 or 3, especially 1 or 2.

Preferably q represents 1 or 2, especially 1.

Preferably (p+q) represents 2, 2 or 4, especially 2 or 3.

Preferably the ring symbol represents a benzene ring.

In relation to the third aspect of the invention we only claim those compounds of general formula I which are novel. We are aware that existing processes only prepare compounds of this type wherein alkyl groups R are of low molecular weight or of high molecular weight; not compounds in which alkyl groups R are of intermediate molecular weight.

Thus, we make no claim to any compounds already disclosed. We know the following compounds to have been disclosed:

compounds having 1 or more alkyl groups R, the or each such group R having at least 9 carbon atoms;

the mixed substance being the sodium salt of mono C₅₋₁₂ alkylbenzene sulphonic acids, as identified in CAS Registry number 68608-87-7 (however, we claim as novel individual non-disclosed sulphonic acid salts embraced by that definition);

diisopropylbenzene sulphonic acid and known derivatives;

toluene sulphonic acid and known derivatives;

2,4-diethylbenzene sulphonic acid;

xylene sulphonic acid and known derivatives;

cumene sulphonic acid and known derivatives.

We may define preferred novel sulphonic acids and derivatives as being C₄-C₈ alkylbenzene sulphonic acids and derivatives thereof (The carbon atom numbers denoting the number of carbon atoms in a single alkyl group when only one alkyl group is present, and the total number of carbon atoms in the plurality of alkyl groups, when more than one is present); Other than diisopropylbenzene sulphonic acid and known derivatives thereof and 2,4-diethylbenzene sulphonic acid. Said derivatives of the novel sulphonic acids of the invention may include metal salts, and optionally substituted ammonium salts, both as discussed previously.

Compounds of the present invention are useful as surfactants and hydrotropes. In particular, alkylbenzene compounds having 5 or more carbon atoms in alkyl group(s) are believed to be effective as surfactants; alkylbenzene compounds having up to 7 carbon atoms in alkyl group(s) are believed to be effective as hydrotropes; and intermediate compounds having 5 to 7 carbon atoms in alkyl group(s) may exhibit surfactant and hydrotropic properties.

The invention will now be further illustrated with reference to the accompanying examples.

Unless indicated otherwise, all examples were carried out using the laboratory equipment described an follows:

A round-bottomed flask was fitted with a polytetrafluoroethene (PTFE) stirrer, driven by an air operated Motor, A reflux condenser fitted with a Dean & Stark dense phase separator was used to collect water from the condensate stream. Also fitted were a dropping funnel for the oleum addition, and a thermometer to measure the boiling range of the reaction mixture. Minor modifications were made as required to allow the system to operate at reduced pressures, in some examples.

In each case the sulphonating reactant was oleum 20%, that in, sulphuric acid containing 20% by weight of SO₃. Molar quantities are expressed below in terms of equivalent sulphuric acid quantities.

Neutralisation can be carried out in any suitable vessel, fitted with an agitation device. Final solvent removal was carried out in a simple laboratory distillation rig.

The unoptimised yield (calculated at the sulphonic acid stage) from the examples below was in excess of 75% and was determined in each case by simple titration.

Compounds referred to hereinafter may be mixed products, e.g. xylene and diisopropylbenzene (the latter as used in the Examples believed to be predominantly the 1,2- and 1,4- isomers). The sulphonated compounds are believed predominantly to have a single sulphonate substituent, probably para- to an alkyl substituent, but this will not be exclusively the case.

### EXAMPLE A

294 g of n-hexylbenzene (1.81 moles) was weighed into the flask, and 118 g of n-heptane added. 183 g of oleum 20% (1.94 moles) was added dropwise with agitation, such that the temperature after the addition was 80° C. The mixture was heated, and refluxed vigorously at 110-115° C. for a hours, until the sulphuric acid content of the sulphonic acid layer was c. 3% by weight. 25 cm³ of water was collected during the reaction. 198 g of water was added to quench the reaction, and the n-heptane recovered by atmospheric distillation at 90-110° C. The sulphonic acid product was a dark brown liquid, containing 65% by weight n-hexylbenzene sulphonic acid and 1.85% by weight sulphuric acid.

The sulphonic acid was neutralised by addition to a dilute solution of sodium hydroxide. This gave a pale yellow solution containing c. 30% by weight of sodium n-hexylbenzene sulphonate. The product was observed to have surfactant properties, producing a semi-stable foam, and producing toluene/water emulsions.

### EXAMPLE B

326 g of diisopropylbenzene (2.01 moles) was weighed into the flask, and 131 g of n-heptane added. 203 g of oleum 20% (2.16 moles) was added dropwise with agitation, such that the temperature after the addition was 80° C. The mixture was heated, and refluxed at 115-118° C. for 2 hours, until the sulphuric acid content of the sulphonic acid layer was c. 4% by weight. 17 cm³ of water was collected during the reaction. 220 g of water was added to quench the reaction, and the n-heptane recovered by atmospheric distillation. The sulphonic acid product was a dark brown liquid, containing 65% by weight diisopropylbenzene sulphonic acid and 2.5% by weight sulphuric acid.

The sulphonic acid was neutralised by addition to a dilute solution of ammonia. This gave a pale yellow solution containing c. 40% by weight of ammonium diisopropylbenzene sulphonate, The product was observed to have surfactant properties, and to behave as a hydrotrope.

### EXAMPLE C

In a modification of Example B, the sulphonation step was carried out under reduced pressure, such that the boiling point of the reaction mixture was 65-70° C. This may be useful to minimise unwanted side reactions (such as sulphone formation, or degradation of the other reagents), It was observed that the reaction race was reduced by the use of vacuum, although a yield in excess of 75% could still be achieved after sufficient time.

### EXAMPLE D

In a modification of Example B, diisopropylbenzene sulphonic acid was prepared in a similar manner. Following the addition of the quench water, the crude acid and solvent were transferred to a separating funnel. It was observed that the n-heptane layer, containing unreacted diisopropylbenzene, formed a separate layer, on top of the aqueous sulphonic acid. The sulphonic acid was run off, allowing the n-heptane and unreacted diisopropylbenzene to be recovered and recycled.

The sulphonic acid was neutralised by addition to a dilute solution of sodium hydroxide to give a solution containing 50% by weight sodium diisopropylbenzene sulphonate. Surprisingly, this was stable to less than 10° C. The solution was dried in a laboratory oven at 110° C., and the resulting mass ground to fine powder The product was readily soluble, and slightly hygroscopic.

A portion of this product was dried in a commercial pilot-scale spray giving a fine, low-density surfactant powder.

### EXAMPLE E

In a further modification of Example B, the process described in Example D was repeated. Prior to neutralisation, it was observed that the alkylbenzene sulphonic acid contained between 1-5% by weight unreacted alkylbenzene. Where the product is to be dried on a commercial scale, this is undesirable. Such quantities would give rise to a risk of fire or explosion in the drying plant, or to the emission of high levels of volatile organic compounds (VOCs).

To minimise the levels of free alkylbenzene, the sulphonic acid was washed with cumene, added in a sufficient amount to form a separate layer Following mixing, the cumene layer was separated from the sulphonic acid layer, using a separating funnel. The separated cumene contained most of the unreacted diisopropylbenzene.

Following neutralisation (as in Example D), the residual cumene (that which had dissolved in the sulphonic acid layer, and was subsequently emulsified in the sodium salt), was removed by distillation under atmospheric conditions. The final product contained less than 0.05% by weight-free alkylbenzene. The process was also observed to significantly reduce sulphone impurities.

### EXAMPLE F

In a prototype industrial process, aqueous diisopropylbenzene sulphonic acid was successfully prepared using a process similar to the examples above on production scale equipment.

Washing of the sulphonic acid was achieved using a production scale liquid-liquid extraction column. This is a packed column, using a stream of xylene flowing countercurrent to the aqueous sulphonic acid.

### EXAMPLE G

2170 g of diisopropylbenzene (13.4 moles) was weighed into the flask, and 870 g of n-octane added. 1355 g of oleum 20% (14.4 moles) was added dropwise with agitation, such that the temperature after the addition was 80° C. The mixture was heated, and refluxed at 134-142° C. for 21 hours, until the sulphuric acid content of the sulphonic acid layer was 0.1% by weight. 190 cm³ of water was collected during the reaction. 1469 g of water was added to quench the reaction. The sulphonic acid product was a dark brown liquid, containing 65% by weight diisopropylbenzene sulphonic acid and 0.7% by weight sulphuric acid. The n-octane/diisopropylbenzene layer was separated for recovery and recycle, ad the aqueous acid washed with excess cumene, to remove residual alkylbenzene and sulphone.

The sulphonic acid was neutralized by addition to a dilute solution of sodium hydroxide. This gave a pale yellow solution containing c. 50% by weight of sodium diisopropylbenzene sulphonate. Cumene therein was removed by atmospheric distillation. The solution was dried in a laboratory oven.

It was observed that the reaction rate at the sulphonic acid stage was higher than an equivalent reaction using n-heptane. It was also observed that the reaction rate could be increased further by the use of reduced pressure.

### EXAMPLE H

407 g of diethylbenzene (3.04 moles) was weighed into a flask and 122 g of n-heptane was added. 304 g of oleum 20% (3.25 moles) was added dropwise with agitation, such that the temperature after the addition was 80° C. The mixture was heated to reflux, and refluxed for 8 hours at 115-122° C., until the sulphonic acid layer contained approximately 2.5% by weight sulphuric acid. 38 cm³ of water had been collected. 289 g of water was added to quench the reaction. The sulphonic acid product was a dark brown liquid, containing 61% by weight diethylbenzene sulphonic acid and 1.4% by weight sulphuric acid.

The sulphonic acid was neutralized by addition to a dilute solution of sodium hydroxide. This gave a dark brown paste containing c. 63% by weight of sodium diethylbenzene sulphonate, The solid was dried in a laboratory oven.

### EXAMPLE I

457.6 g of n-octyl benzene (2.4 moles) was weighed into the flask, and 137.3 g of n-heptane added. 244.6 g of oleum 20% (2.6 moles) was added dropwise with agitation, such that the temperature after the addition was 80° C. The mixture was heated, and refluxed at 110-117° C. for is hours, until the sulphuric acid content of the sulphonic acid layer was c. 2.7% by weight. 35 cm³ of water was collected during the reaction. 297.7 g of water was added to quench the reaction. Luring this stage the material become gel like and foamed excessively. The sulphonic acid product wax a dark brown liquid containing 60% by weight n-octylbenzene sulphonic acid and 2.1% by weight sulphuric acid.

The sulphonic acid was neutralised by addition to a dilute solution of sodium hydroxide. This gave a pale brown viscous paste containing c. 50% by weight of sodium n-octylbenzene sulphonate. The paste was dried in a laboratory oven.

Surfactant like properties of the material were observed.

### EXAMPLE J

1481.3 g (15.8 moles) of phenol was weighed into the flask and 518.3 g of n-heptane added. 1472.5 g of oleum 20% (15.7 moles) was added dropwise with agitation, such that the temperature after the addition was 80° C. The mixture was heated, and refluxed an 100-103° C. for 5.5 hours, until the sulphuric acid content of the sulphonic acid layer was c. 1% by weight. 50 cm³ of water was collected during the reaction. No quench water was added at this stage. The sulphonic acid product was a dark brown liquid, containing 83% by weight phenol sulphonic acid and 1.2% by weight sulphuric acid. The free phenol level at this stage was 0.2% by weight.

The sulphonic acid was neutralised by addition to a dilute solution of sodium hydroxide. This gave a pale grey paste containing approximately 50% by weight of sodium phenol sulphonate. Dissolved solvent was removed by atmospheric distillation. The solution was dried in a laboratory oven.

The reader's attention is directed to all papers and documents which are filed concurrently with or previous to this specification in connection with this application and which are open to public inspection with this specification and the contents of all such papers and documents are incorporated herein by reference.

All of the textures disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A process for the sulphonation of an organic compound, which process comprises reacting the organic compound with sulphuric acid or oleum in the presence of an organic solvent at an elevated temperature.

2. A process as claimed in claim 1 wherein the solvent is present as 15-70% of the total weight of the reaction mixture.

3. A process as claimed in claim 1 or 2, wherein the solvent is an alkane or cycloalkane.

4. A process as claimed in claim 3, wherein the solvent is an n-alkane having 4-14 carbon atoms.

5. A process as claimed in any preceding claim, wherein the organic compound is a hydroxybenzene or an alkylbenzene compound.

6. A process as claimed in claim 5, wherein the organic compound is an alkylbenzene compound having one or more alkyl groups which have in total 4-8 carbon atoms.

7. A method of extracting an alkylbenzene sulphonic compound from a sulphonation reaction mixture containing unreacted alkylbenzene, wherein the benzene group carries one or more alkyl groups comprising in total n or more carbon atoms wherein n is at least 3, which process comprises contacting the reaction mixture with a second, purifying, alkylbenzene compound having one or more alkyl groups comprising in total no more than m carbon atoms, wherein n is two or more integers greater than m, the purifying compound being able to form a separate layer in the reaction mixture, wherein the purifying compound is provided in an amount sufficient to form a said separate layer; and physically separating the two layers to remove unreacted alkylbenzene partitioned within the purifying compound layer.

8. A method as claimed in claim 7, applied to the extraction of the desired product of a sulphonation process as claimed in any of claims 1 to 6, wherein the reactant organic compound is an alkylbenzene compound having one, two or three alkyl groups which comprise 3-14 carbon atoms in total, and the second, purifying, alkylbenzene compound has one or two alkyl groups which comprise 1-3 carbon atoms in total.

9. A method as claimed in claim 8, wherein the second, purifying, alkylbenzene compound is selected from cumene, xylene and toluene.

10. A compound of general formula 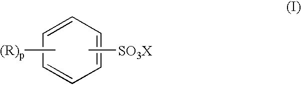
wherein R represents, or each group R independently represents, an optionally substituted alkyl or hydroxyl group, X represents a hydrogen or metal atom or an optionally substituted ammonium group, and p represents 1, 2 or 3; but not including; compounds having one or more alkyl group R, the or each such group R having at least 9 carbon atoms; the mixed substance being the sodium salt of mono C₆₋₁₂ alkylbenzene sulphonic acids;
diisopropylbenzene sulphonic acid and known derivatives;
toluene sulphonic acid and known derivatives;
2,4-diethylbenzene sulphonic acid;
xylene sulphonic acid and known derivatives;
cumene sulphonic acid and known derivatives.

11. A compound as claimed in claim 10, being a C₄-C₈ alkylbenzene sulphonic acid or a derivative thereof i(the carbon atom numbers denoting the number of carbon atoms in a single alkyl group when only one alkyl group is present, and the total number of carbon atoms in the plurality of alkyl groups, when more than one is present); other than diisopropylbenzene sulphonic acid and known derivatives thereof and 2,4-diethylbenzene sulphonic acid.

12. A compound as claimed in claim 11 comprising an alkylbenzene sulphonic acid or derivative thereof, the benzene ring carrying an n-alkyl group having 4-5 carbon atoms.

13. A compound prepared by a process as claimed in any of claims 1 to 6 or by a method as claimed in any of claims 7 to 9.
